(19) United States
(12) Patent Application Publication
(10) Pub. No.: US 2014/0194774 A1
(43) Pub. Date: Jul. 10, 2014

(54) SYSTEM AND METHOD FOR HEARING ASSESSMENT OVER A NETWORK

(71) Applicant: Robert Gilligan, San Francisco, CA (US)

(72) Inventor: Robert Gilligan, San Francisco, CA (US)

(21) Appl. No.: 13/738,962

(22) Filed: Jan. 10, 2013

(51) Int. Cl. A61B 5/12; A61B 5/00

(52) U.S. Cl. CPC A61B 5/123; A61B 5/0022; A61B 5/7203; A61B 5/7405
USPC 600/559

(57) ABSTRACT

A system for administering a hearing test to a remotely located user or subject using a user computing device connected to a communication network. The system provides for testing of ambient noise at the remote location and accuracy of sound reproduction by headphones engaged to the user computing device in a calibration of the loudspeakers of the headphones and the venue prior to communicating the test sounds. The calibration insures results determined by user input provide an accurate outcome.

### BACKGROUND OF THE INVENTION

1. Field of the Invention

The disclosed device and method relate to the testing of a patient's sense of hearing. More particularly, the disclosed system relates to a system and method for the evaluation of the sensitivity of a person's sense of hearing, where the person is in a location remote to the testing facility. A location that can be even hundreds of miles remote. Using a system which accommodates room and local noise generated at the locale of the patient, both the patient testing and communication of their results, may be accomplished employing networked communication between the patient over a server employing communications software adapted to both tasks. The testing may be accomplished with or without concurrent input from a hearing professional.

2. Background of the Invention

According to data reviewed by the National Academy On An Aging Society about 8 percent of the United States population have impaired hearing. This percentage of the population numbers more than twenty million Americans who suffer from some form of hearing impairment which may vary from mild loss of hearing and sensitivity to total loss of hearing. It is also estimated that such hearing impairments, left untreated, cost the U.S. economy $56 billion annually due to lost productivity, special education, and medical care. In addition, it is known that there is a direct correlation between hearing loss and the age of a patient. Since the population of the United States and many countries is on average getting older, hearing problems amongst the general population will only increase in the future.

In order to ascertain, and subsequently treat a patient's hearing loss, conventionally a hearing test is conducted. Such tests are conducted by an audiologist or other hearing professional who supervises the testing. A hearing test provides the reviewer and subsequently the patient, an evaluation of the sensitivity of the tested person's sense of hearing. Such tests are conventionally performed by a trained audiologist employing an audiometer.

Audiometers are standard equipment for hearing tests and they conventionally consist of an embedded hardware unit connected to a pair of headphones which communicate tones and sounds to each ear of a patient. During such tests a patient feedback button is provided for the patient to indicate what or if they are hearing the sound in the headphones. Audiometer requirements and the test procedure are specified in IEC 60645, ISO 8253, and ANSI S3.6 standards which are incorporated herein by reference.

Employing such an audiometer the audiologist is provided with a standardized manner for evaluating a person's hearing sensitivity at different frequencies of sound. Using the audiometer and headphones, after a calibration has been conducted, the audiologist can perform an accurate evaluation across the generated frequencies.

An audiometer hearing test is conventionally administered to a person sitting in a quiet room or substantially soundproof testing area. The patient being tested wears a set of headphones during the test, which is also in communication with an audiometer. Over-the-ear or headphones covering the entire ear area are frequently employed. Headphones having small foam inserts which are positioned in the ear canal, often referred to as earbud type headphones, are also used.

Such tests are conventionally conducted at a facility managed by the hearing professional for a number of reasons. A primary reason is that any audiometer which is employed for testing of hearing, must be calibrated regularly. Calibration insures that the sound level and frequencies shown on the display connected to the audiometer, is equal to the actual sound stimulus to which the testing subject is being exposed. Further, such testing is handled in professional facilities to insure that virtually no background noise is being communicated to the testing subject who may be placed in a soundproof room during the testing.

Accurate and reliable test measurements of each patient or subject, are a major component in characterizing and quantifying any test subject's hearing loss. Only with proper calibration of the equipment and elimination of background noise, can the hearing professional insure that the measurements taken of each patient or test subject, are consistent for all patients. In this fashion, using test results, hearing aids for the test subject can be programmed to accommodate the sound frequencies and levels which are deemed impaired in the test subject. Background noise interfering with the results or uncalibrated test equipment will yield poor results.

During the hearing test, the audiometer communicating sound signals to the headphones, causes them to produce tones of sound at specific frequencies which are communicated by the loudspeakers of the headphones to one or both of the test subject's respective ears. Frequencies and volume levels may be set and communicated to each ear independently allowing the tester, using calibrated equipment and room surroundings, to discern the strengths and weaknesses of the hearing of each of the test subject's ears, independent of the other.

During the test, the audiologist or licensed hearing specialist, plots the loudness in decibels and the various frequencies of tones generated and communicated by the headphones, and this plotting yields an audiogram. During the test, patients being tested will convey to the tester whether or not they have heard the tone conventionally through signaling with a hand or pressing the feedback button.

As the test progresses over the various frequencies and at volumes communicated to each respective ear, the audiologist or hearing specialist will plot points on the graph, where conventionally the frequency is plotted on the x-axis and the loudness is plotted on the y-axis. Once each frequency of hearing ability is tested and plotted, the points are fitted to a line so that the patient and professional can ascertain which sound frequencies are not being heard normally by the test subject, and to what degree of hearing loss the tested person may suffer. Once the patient's weak points are ascertained for each ear, using this standardized test with properly calibrated equipment and surroundings, hearing aids may be custom tuned to the patient's individual hearing strengths and weaknesses. This is because hearing aid manufacturers generally construct the hearing aids to norms established from the standardized hearing test results. Thus, they may be adjusted to digitally enhance the frequencies where a test subject lacks proficient hearing.

A complete hearing evaluation of a patient with hearing loss can employ other tests as well. These may be conducted to ascertain what type of hearing loss is present and may involve tuning forks which are used to determine if there is a conductive hearing loss caused by problems with the outer or inner ear, a sensorineural loss caused by problems in the cochlea, or neural loss caused by a problem in the auditory nerve or auditory pathways of the brain. Further, the audiologist or hearing instrument specialist may also conduct speech tests, wherein the patient repeats the words he or she hears.

Once testing is finished using the properly calibrated equipment and the resulting audiogram, the patient is fitted for new hearing aids if needed. As noted, using the industry norms based on standardized audiograms with calibrated equipment, the newly fitted or existing hearing aids can be adjusted based upon the recent hearing test to provide the most help in the frequency ranges where the patient has the most impairment. This is especially true of new multichannel digital hearing aids which may be specifically tuned to provide the patient with volumes at frequencies where they need the most help.

However, a number of problems exist in this scenario. First, for reason noted, testing of individuals conventionally must be handled in a lab setting at a medical facility or office with an audiologist or other professional present who is certified in the country or state in which the test is performed. However the uses of professional locations and requirement for a professional being present during a test, severely limits the number of tests possible in a given time period by the number of such testing facilities located in a geographic area. Further limiting such testing is the limited number of hours such facilities are open and the hours that the hearing professional are able to work.

Another major problem is one of the patient's denial of a hearing problem or one of a patient's failure to discern they even have a problem. Since hearing loss, in most cases, is very gradual, the person with a moderate hearing loss may never have noticed they were becoming hearing-impaired due to the gradual declination of their hearing. Other patient's may be younger and have not even considered the possibility of hearing loss. Consequently, a large portion of the population with moderate to worse hearing loss is either in denial or fails to ascertain that they even have the problem. In either case, making the conscious decision to visit the offices of a hearing professional and making an appointment to do so, is out of the question.

As a consequence of limited facility availability, limited hours hearing professionals are available, patient denial, and further coupled with the inability of many individuals to admit or discern a hearing loss, a large portion of the population suffering from hearing loss that can be adequately treated, are simply not tested. People who are unaware they have a problem are unlikely to seek testing especially where appointments are limited to testing times which are months in advance due to limited facilities. Patients who won't admit the problem are just as unlikely to actively seek testing where they must do so in advance, take the effort to make an appointment, and then travel to a geographically remote location from their home. This is especially true of older patients who may have limited driving capability.

As such, their exists a continuing and unmet need for a system to allow patients to be accurately tested for hearing loss in the comfort of their home or office, or another location, remote from conventional hearing test centers. Such a system should, at least initially, be free of a requirement of participation from the audiologist or hearing professional but should be configured to yield accurate results with calibrated equipment. Such a system should endeavor to provide accurate results by providing a system where test subjects may be tested using communication over a network such as the internet, and employing software adapted to the task of calibrating the equipment and accommodating for local noise, to discern remotely, the actual tone and frequencies which are and have been communicated from the patient's headphones remotely to the patient's ears, without having a medical professional physically be present, to yield results similar to tests performed in professional centers. Such a system should provide results which are discernable by software adapted to the task of reviewing test results and/or by a reviewing professional, and which provide the ability to show the areas of hearing loss on the spectrum where a patent is impaired.

Such a system, allowing for remote or in-home testing, will also provide the user or patient the opportunity to ascertain if they might have a hearing impairment, prior to testing at a professional office, to thereby induce the patient to admit or understand their hearing problem sufficiently to request testing. Finally, such a system, in an optional mode, subsequent to testing, and employing software adapted to the task of analyzing the test outcome and adjusting earphone output, should provide a means for the patient to experience a virtual hearing enhancement from a virtual hearing aid session, to thereby educate the patient on the potential for correction of any hearing impairment. Optionally, such a system should also be able to employ software adapted to make or direct a test subject to make an adjustment of their existing hearing aids, subsequent to remote testing, to allow for changes to hearing aid settings over time to accommodate the patient or user's hearing changes.

The forgoing examples of related art and limitation related therewith are intended to be illustrative and not exclusive, and they do not imply any limitations on the invention described and claimed herein. Various limitations of the related art will become apparent to those skilled in the art upon a reading and understanding of the specification below and the accompanying drawings.

### SUMMARY OF THE INVENTION

The invention herein disclosed and described provides a solution to the shortcomings in prior art and achieves the above noted goals of achieving accurate hearing tests in the comfort of the users home or office through the provision of a system and method employing computer hardware with networking capabilities, and running software adapted to the various tasks to provide a conventional hearing test previously noted which will yield results which are employable to discern hearing loss in the test subject, and for hearing aid adjustment for hearing aids which are adjustable according to industry standard test results.

Employing the software running on a networked server and in communication with a computer device proximate to a test subject with earphones engaged with the computer device, they system herein using results therefrom can also provide the remote test subject through the earphones worn an example of a virtual hearing aid which when listened to would accommodate their hearing loss, as well as be able to synthesize hearing impairment simulations for a mate of the test subject to understand the problem of the test subject.

Optionally but preferred, the system employing software adapted to review the hearing tests of the subject, and to adjust a brand of hearing aid according to manufacturer's specifications for the tested impairment, will make or inform the test subject how to make hearing aid adjustments. The information passed to the test subject or the adjustment to the hearing aid will be performed over a wide area network such as the Internet using software which either or both produces instructions for the test subject to make adjustments, or communicates directly with the hearing aid and makes adjustments.

The system, thus, alleviates the need for visiting and use of the noted limited number of testing facilities available to patients or users of a geographic area by enabling the patient and users to test themselves in the comfort of their home or office. In addition, for patients in remote areas of a country, where no hearing professionals are located within a reasonable travel range, the system provides a means for remote users to obtain hearing tests, and hearing aid adjustments where they might otherwise be precluded from such.

As noted, in conventional testing settings, the audiologist or trained professional employs headphones with a known and a tested output of sound and frequency and volume to the person being tested resulting from the signals communicated electronically to those headphones. Only with a known sound, volume, and frequency being delivered to the patient's ear, can the professional testing the patient determine what sounds, tones, or frequencies that patient has trouble discerning. Without a standard of known sound production being delivered, no reasonable accuracy to the test can be guaranteed. These requirements of a known sound delivery, and an administering professional, are a major factor limiting the testing of patients, since conventionally at testing facilities, the audiologist or tester must check the equipment communicating the sound to the patient being tested during the test.

However, with the development of digital signal processing (DSP) for sound, versus the analog mode, employing sound generating equipment and computers running software adapted to the task, a remote calibration of headphones being employed by the user or patient being tested, can be ascertained. Such can be employed using software adapted to the task of calibrating and testing, to allow for testing without an audiologist or trained professional being present.

Thus, employing the system herein and software adapted to operate over a network to remotely calibrate the patient's connected headphones, and sample room noise and accommodate it in testing and results, professional testing results are yielded which may be employed to discern hearing loss and/or adjust hearing aids or provide virtual hearing loss sessions to mates of the test subject.

In the system which operates employing software running from a computing device with communicating memory and which communicates over a network using software configured to administer conventional hearing tests conducted by a hearing professional, patients can be tested over the internet or another network communicating between the patient being tested and the remote server computing device. Optionally, the user or patient can be referred to an audiologist to further quantify test results and/or recommend hearing aids or adjustments thereto. Such referral can be accomplished by communicating of the need for further testing to audiologists or other testing professionals over the network. This can be in the form of a text communication, for example.

Currently, the system allows the remotely located user, test subject, or patient being tested to employ their own headphones operatively connected to a computer capable of having hardware and software configured for generating sounds based on electronic signals communicated thereto over the network from the remote computing device or server of the tester. The actual sound produced by the patient's headphones in the remote location, and thus the frequency and tone communicated to their ears during the test, is calibrated to reach test required norms using a microphone at the remote site of the user or test subject.

The sounds generated by the software on the server running the test software and communicated over the network to the headphones, are sampled by the microphone which is operatively engaged to the computer to which the headphones are engaged. Sound samples are taken and recorded to memory from the headphones through the microphone or transmitted directly back to the server running the test.

If recorded, the sound recordings may be sent to the server running the test and are analyzed for the reproduction produced by the headphones, using software as noted below which analyzes electronic sound files to ascertain the exact frequency and tone produced remotely. Software running on the remote computer of the user might also be employed for this task, and the results communicated back to the server running the test.

Using the sound files from the loudspeakers reproduction from the headphones, and employing software configured to analize of that reproduction, adjustments can be made on the server running the testing software such that subseqent transmitted sounds from the server running the test software, are adjusted to yield sound eminating from the speakers of the headphones, which matches the required tone and frequency and any other sound characteristics required for a standardized test of a test subject.

As noted, the microphone receiving sound emanating from the headphone speakers, can either communicate it directly back over the network in an analog or other appropriate signal to the server running the testing software routine for analysis. Or, If sound files are recorded, communicated and analyized may be MIDI or WAV, AIFF, AU or raw header-less PCM or MP3, MP4, ALPC, or some other preferably lossless file format which can be read and reproduced accurately and analyized in computer memory using the appropriate Codec, and software configured to review the sound recorded through the microphone from the remote headphones.

Currently a preferred mode for running the real time calibration over the computer network is by employment of a dual FFT (fast-fourier transform) in combination with audio analysis software adapted to the task of analyzing the sound received back as an analog or digital signal, or as a file. Conventionally, discrete or Fast Fourier Transform converts a finite list of equally-spaced samples of a function, into the list of coefficients of a finite combination of complex sinusoids, ordered by their frequencies, which have those same sample values. It can be described as converting the sampled function from its original domain (often time or position along a line), to the frequency domain.

The input samples are complex numbers (in practice, usually real numbers), and the output coefficients are complex too. The frequencies of the output sinusoids are integer multiples of a fundamental frequency, whose corresponding period is the length of the sampling interval.

The combination of sinusoids obtained through the DFT is therefore periodic with that same period. The DFT differs from the discrete-time Fourier transform (DTFT) in that its input and output sequences are both finite; it is therefore said to be the Fourier analysis of finite-domain (or periodic) discrete-time functions.

The DFT is the most important discrete transform, used to perform Fourier analysis in many practical applications such as herein. In digital signal processing, the function is any quantity or signal that varies over time, such as the sound wave signal emanated from the headphones to the microphone, and sampled over a finite time interval (often defined by a window function).

Since it deals with a finite amount of data, DFT and FFT analysis can be implemented employing software in computers with numerical algorithms or even dedicated hardware. These implementations usually employ efficient fast Fourier transform (FFT) algorithms, so much so that the terms “FFT” and “DFT” are often used interchangeably.

Using FFT analysis software working in combination with the sound generation transmission of the test software running on the server, the transmitted sound signal can be adjusted to produce sound signals from the headphones, which match a predetermined norm which would be stored in memory. The returning sound signal representing the broadcast sound from the headphones, is adjusted by adjusting the transmitted sound production signal, until the returning signal shows a match to test norms thereby showing proper calibration.

At the start of the test, text instructions are communicated from the server for display on the user's remote screen and/or voice instructions may be transmitted for play through the user's computer and will instruct the user to be quiet. The user or patient will be asked to send a signal using an input device such as a mouse or keyboard key, that they are quiet. Once the instruction is received and acknowledged by the patient, the software running the test on the server remotely will cause the microphone to turn on and to take a reading of any ambient noise communicated to the microphone, at the remote location of the user or patient.

The testing software running on the server or remote machines will receive a signal over the network representative of the sound captured by the microphone either directly or using a recorded digital file as noted above. Again employing software configured for conducting an FFT analysis, the captured sound of ambient noise will be examined. The FFT analyzer routine, in similar fashion to analyzing transmitted and received sound signals, removes time from the captured clip, and transforms it into a virtual frequency amplitude graph which may be stored on the user's remote computer in memory, or on the server running the test software.

Initially, the system assumes that the captured ambient noise is at an amplitude of 30 db, and employing the stored graph produced by FFT analysis, this assumption can be affirmed employing the software configured to the task to examine the graph file of the noise, and ascertain there are no major peaks or troughs in the graph file which would cause the 30 db assumption to be questioned.

If it is determined by software analysis that the sound graph confirms the assumption of a 30 db background noise, a next step in the system is initiated by the software. It is noted that in other modes the system may be configured to assume a higher or lower decibel level when analyzing the ambient noise as deemed suitable by the designer. Currently, the 30 db assumption is given as a preferred initial assumption as it has worked effectively in testing.

In this step, the user is instructed to test the headphones for actual projection of sound. The user or patient will be given video and/or audio instructions to test the headphone output by positioning of the headphones adjacent to a microphone operationally communicating with their computer. The user is instructed to sequentially position each of the two speakers of the headphones adjacent to their microphone, and then use an input device on their computer to confirm positioning. This can be done by pushing a button on their computer or mouse.

During each testing of the output of each headphone speaker, software running the system and communicating with the user or patient's computer generates a signal which is transmitted to the test subject or patient's computer which is calculated to produce a substantially pure tone at preferably 500 hertz. Other suitable frequencies may be employed however 500 hertz has shown to perform well in testing.

This tone is communicated sequentially from each headphone speaker, to the adjacent placed microphone. The tones generated can also include volume increases to accommodate assumed noise and positioning distances from the microphone and for additional testing data by increasing in increments, for example in 20 db increments up to 60 db.

The microphone communicates the received samples of the pure tones generated by the respective headphone speakers from one or a plurality of distances therefrom during the test performed by the user or patient following the communicated instructions from the server through their computer.

Software adapted to the task on the server or running on the computer communicating with the server, segments the communicated tone segments into multiple samples of, for instance, 50 ms in length, to thereby generate graph points which the software overlays on a graph of the communicated sounds received from the user's microphone, from those representative of the communicated to and generated by the headphones.

This graph overlay, when averaged, provides the software running the system gross functions allowing for an FFT analysis of the actual sounds generated at the user or patient's site, using their headphones and microphone. The FFT analysis coupled with software adapted to the task provides the means for adjusting the transmitted sound signal for calibrating the remote headphones to generate the reference sound at substantially the correct tone and volume and insure the sound heard by the patient or user, is as close as possible to the tones, and frequencies of the original transmitted material. More accommodations can be made in the generated and communicated frequencies and sounds if necessary and a subsequent calibration conducted to ascertain sound generation by the user or patient's headphones within professional standards.

Thus, the patient or user located in a substantially quiet room, across town or across the country, can have a test conducted of their hearing with substantial accuracy as to the results since the software performing the test is adapted to ascertain that the sound being communicated to the patient's ears through their headphones is calibrated and correct. During the calibration sequence, the patient would employ a mouse button or key from the keyboard, or other means of input signaling as instructed by one or a combination of visual cues and auditory cues transmitted to them during the test. Visual cues may be using indicia on the video screen of the computer being used. Auditory cues may be a secondary channel of sound communicated through the earphones.

Subsequent to the calibration phase, software configured for the task running on the server or communicating system computer, can conduct the hearing test with the remote user or patient. Optionally, the system can be configured for an audiologist or other hearing professional to perform the test from a location remote of the user, or aid the software in performing the testing if desired.

In addition, as an option to the user, live or recorded video feed of the user performing the test can be communicated to the hearing professional as to allow the professional to further aid the user in correctly performing the test, or to answer questions the user may have during test performance. This can be accomplished through the employment of video and audio recording means, such as a video or web-enable camera and microphone, which is in communication with the user's computer hardware or with the server over the network, which is configured to send recorded or live video and audio over the network to the professional for immediate or later review. Other means for direct lines of communication can include telephone or cellular network communication.

The conventional hearing test is conducted where the remote patient or user responds to discerned audio communications to each ear, using video cues generated concurrently on their video display which are transmitted by software running the test and generating testing tones on the headphones. During the test, the tones are communicated in individual frequencies through each respective speaker of the headphones to the adjacent ear. During the testing the user or patient is asked by video displayed queries the lowest volume tone of each communicated frequency they can hear from the generated frequency tones communicated at sequentially increasing volumes. Software configured to the task, ascertaining the inputs from the user or patient as to the lowest volume sound they discern at each communicated frequency tone, will ascertain any hearing impairment the communicating user or patient suffers.

It is thus extremely important that during the calibration phase that proper levels of ambient noise be discerned prior to the test and that the headphones be calibrated by the system to generate the actual sound transmission required of the standard test, to the patient or user's ears. As such, if the system employing software configured to the task, detects a mistake or possible headphone mis-calibration either due to hardware error or user error during the calibration process, the user may be prompted to re-calibrate their headphones, calibrate a different set of headphones, or to seek assistance from a conventional testing professional.

Using the patient input to the generated sequential tones and frequencies, the system employing software adapted to the task assembles an audiogram. The virtual audiogram plots points on a graph where the frequency is on the x-axis and the loudness on the y-axis. Once each frequency of hearing ability is tested and, using the remote patient responses, is plotted, the points can be fitted to a line and printed or displayed to allow the user, patient or hearing professional, to ascertain at a glance which frequencies are not being heard normally and what degree of hearing loss may be present with the remote user.

The software running on the system can communicate the audiograph to the user or patient for their use in obtaining a hearing aid, or can be communicated to a hearing professional for review and to provide advice to the user or patient on hearing aids or other measures which may be taken to correct the ascertained hearing impairment.

The system can also be employed by hearing aid manufacturers or providers, to allow for patient or user initial or ongoing communication regarding their products. Employed in this fashion, the hearing aid provider or manufacturer would provide a means for hearing aid communication with the patient or user's computer which would communicate with the server and/or remote computer of the manufacturer or provider. Means for hearing aid communication with the patients computer can be wireless RF communication means such as Bluetooth® or wifi communication, or through a physical wired connection to the users computer.

The test above would be performed and if the patient or user does not have a hearing aid, one could be recommended and/or a local hearing professional selling the manufacturer's products could be forwarded contact information for the user or patient. Using that information the local professional will contact the patient and communicate hearing aids or other options available to the user or patient to aid the hearing impairment discerned in the test. Or, if the user and patient are already in a direct line of communication, such as through live video and audio feed, then recommendations can be given immediately to the user by the professional. This may be preferred if the user had initially ascertained a substantial hearing loss, which is then confirmed by the test, and therefore immediate hearing improvement recommendations will substantially improve the users quality of life.

In accordance with other preferred modes, in countries or areas where patients may be geographically or otherwise precluded from visiting a hearing professional, the system can be employed by such manufacturers or hearing professionals to allow users to make initial and periodic adjustments to hearing aid equipment provided by them. Modern digital hearing aids are adjustable to accommodate the hearing loss of the wearer and many do so using multiple channels of sound generation and amplification. The test herein once performed can be employed by software adapted to the task running on the server, manufacturer's system, or user's system to make adjustments to the sound amplification system of the patient or user's hearing aids. This would be done by placing the hearing aids in communication with the user's computer using wireless RF communication such as Bluetooth® or direct wired communication such as USB or other suitable connection means. Once connected, the software running on the hearing aids would be adjusted using the results from the user or patient's hearing test on the system herein to tune the sound communicated to each respective ear of the wearer to that ear's respective discerned impairment.

Further, since the sound produced by the user or patient's headphones can be first calibrated remotely, to ascertain the accuracy of the sound actually communicated to their ears, other modes of operation of the system may be employed. Such modes may include simulating real life instances from which a person may suffer hearing impairment such as a crowded noisy restaurant or on a street during heavy traffic. Using software adapted to the task, running on the system, the user or patient can then be provided with “virtual” hearing aids where the headphones are adjusted to generate sound in a manner similar to a properly adjusted hearing aid. The user would thus hear the improvement a hearing aid would provide before buying it.

Still further, for the patient or their family, simulated hearing problems may be communicated where the communicated sound from the headphones to the wearer is changed so simulate how the patient actually hears. This type of service could be provided to the patient's family or friends so they can ascertain how the patient actually hears and perhaps better understand the problem and maybe even try to accommodate it in the future.

Finally, the system herein may also be configured to be employed with people who know they have a hearing impairment and already wear hearing aids for adjustment of such. Using the system herein for testing of the hearing aid user, and a means to communicate with the digital hearing aids such as a USB engagement with the patient's computer, the hearing aids can be remotely adjusted to produce sound best adapted to accommodate the discerned hearing impairment of the wearer during the test. This would alleviate the need for hearing aid wearers to drive to distant centers for such adjustments.

With respect to the above description, before explaining at least one preferred embodiment of the herein disclosed invention in detail, it is to be understood that the invention is not limited in its application to the details of construction and to the arrangement of the steps in the following description or illustrated in the drawings. The invention herein described is capable of other embodiments and of being practiced and carried out in various ways which will become obvious to those skilled in the art on reading this disclosure. Also, it is to be understood that the phraseology and terminology employed herein are for the purpose of description and should not be regarded as limiting.

As such and as noted, those skilled in the art will appreciate that the conception upon which this disclosure is based, may readily be utilized as a basis for designing of and operation of other methods and systems for carrying out the remote testing and simulation and several purposes of the present disclosed system. It is important, therefore, that the claims be regarded as including such equivalent construction and methodology insofar as they do not depart from the spirit and scope of the present invention.

It is an object of the invention to provide a web-based system for testing for hearing impairment having a calibration system to ensure similar results to local testing.

It is a further object of this invention to provide such a system which allows the tested person to employ their own headphones which are remotely calibrated to provide accurate test results from a software driven and calibrated hearing exam.

It is an additional object of this invention to provide such a system which will also provide the ability to communicate virtual or artificial hearing impairment situations to the remote user to ascertain hearing loss in different environmental situations.

Yet an additional object of this invention to provide such a system which may also be used by associates of the patient or tested person, to communicate sound to such associates in a fashion which mimics how the patient hears, to allow a better understanding and perhaps compensation by such an associate of the patient.

A further object of this invention is the provision of a system to allow owners of hearing aids to test their hearing and provide employ results generated therefrom to adjust their hearing aids to accommodate the current state of any hearing impairment.

Further objectives of this invention will be brought out and discerned by those skilled in the art through a reading of the following part of the specification wherein detailed description is provided for the purpose of fully disclosing the invention, without placing limitations thereon.

### BRIEF DESCRIPTION OF DRAWING FIGURES

The accompanying drawings, which are incorporated herein and form a part of the specification, illustrate some, but not the only or exclusive, examples of embodiments and/or features. It is intended that the embodiments and figures disclosed herein are to be considered illustrative rather than limiting. In the drawings:

FIG. 1 depicts a diagram of the system herein showing the steps of establishing communication with the user or patient being tested, calibration, and testing.

FIG. 2 shows a graph of amplitude and frequency of sound.

FIG. 3 depicts a graph of a received sound “T” transmitted by a microphone from sound generated by remote headphones, in a comparison to a reference tone “R” stored in computer memory of proper calibration.

FIG. 4 depicts a graph of ambient background noise below a 30 db threshold which is preferred.

Other aspects of the present invention shall be more readily understood when considered in conjunction with the accompanying drawings, and the following detailed description, neither of which should be considered limiting.

### DETAILED DESCRIPTION OF THE PREFERRED EMBODIMENTS OF THE INVENTION

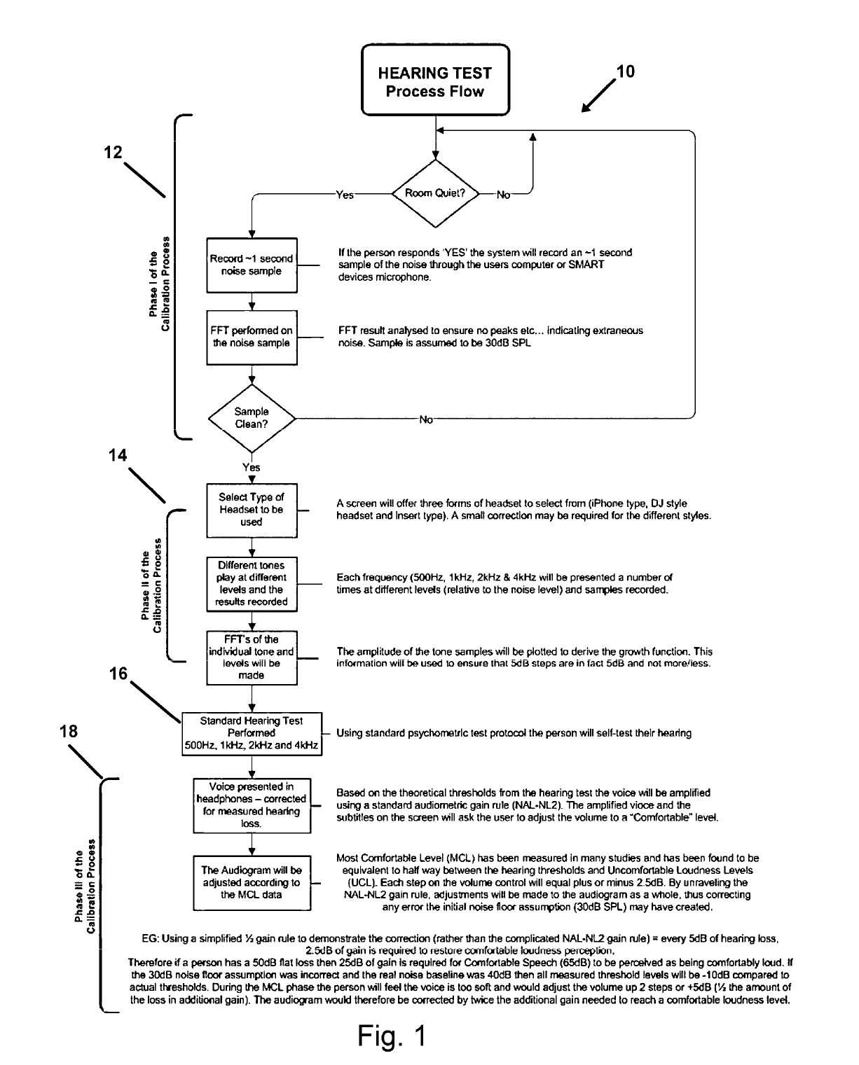

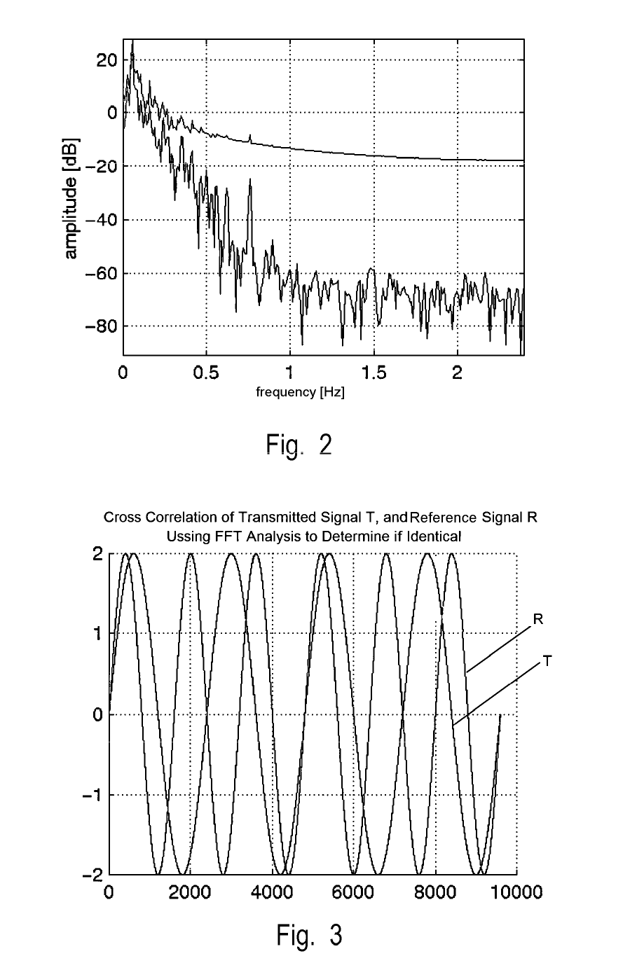

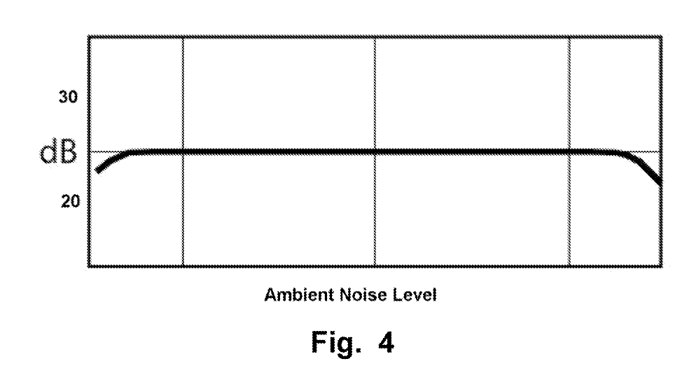

Now referring to the drawings, in FIG. 1, there is shown a block diagram of the system herein generally providing figures and flowpaths representative of the disclosed system 10 for remote testing for hearing impairment.

The system 10 and method herein, in a first step 12 employs electronic means for determination of a maximum allowable db level of room noise in a calibration of the remote location and to ensure the sampled noise levels fit within system norms and a preferred assumption of 30 db threshold noise levels.

At the start of the test, text instructions are communicated from the server for display on the user's remote screen and/or voice instructions may be transmitted for play through the user's computer and will instruct the user to be quiet. The user or patient will be asked to send a signal using an input device such as a mouse or keyboard key, that they are quiet. Once the instruction is received and acknowledged by the patient, the software running the test on the server remotely will cause the microphone to turn on and to take a reading of any ambient noise communicated to the microphone, at the remote location of the user or patient.

The testing software running on the server or remote machines will receive a signal over the network representative of the sound captured by the microphone either directly or using a recorded digital file as noted above. Again employing software configured for conducting an FFT analysis, the captured sound of ambient noise will be examined. The FFT analyzer routine, in similar fashion to analyzing transmitted and received sound signals, removes time from the captured clip, and transforms it into a virtual frequency amplitude graph which may be stored on the user's remote computer in memory, or on the server running the test software.

Initially, the software running on the server or host computer running the test will be configured to assume that the captured ambient noise, is at an amplitude of 30 db. Employing an electronically produced and reviewable graph produced by FFT or other comparable analysis, this assumption can be affirmed. This is done by employing a routine of the testing software configured to the task of examining the produced graph file representing the noise generated from signals captured by a microphone at the remote location, and ascertaining there are no major peaks or troughs in the line of the graph file which would cause the 30 db assumption to be questioned.

If it is determined by software analysis that the sound graph generated using the microphone sounds of ambient noise at the remote location, confirms the assumption of a maximum of a 30 db background noise, the next step in the system is initiated by the software.

In a second phase of the system, calibration of the headphones to discern and adjust their actual reproduction of sound transmitted to the ears of the remote user or patient is ascertained. Optionally, the user or patient may be queried, using transmitted video and/or text which displays remotely on their computer screen, as to the nature or brand of the headphones they will employ. This can shorten the test since knowing the qualities of the headphones and storing such in a lookup table can allow the software for the testing to make initial adjustments in transmitted sound signals.

If the user is unable to provide adequate information about their headphones, the user may be able to select exact manufacturer make and model of the headphone being used, therefor allowing the system to obtain headphone information based of manufacturer specifications which may be stored in a database on the sever, or the system may employ software adapted to the task of conducting an internet search based of the user inputted make and model to find such information.

Whether or not the headphones are identified, thereafter, signals calculated to produce selected tones at selected frequencies from the headphones are communicated over the network for reproduction by the headphone speakers. With the patient or user being directed to place the microphone adjacent to each respective headphone speaker, the tones are communicated to the microphone of the user or patient from each respective headphone speaker. Samples may be taken at one position or by placing the headphone at one or a plurality of predetermined distances to the microphone.

Electronic signals representative of the received sound, from the microphone, of the user patient's computer are communicated over the network and returned to the server or computer running the testing software configured to run the system herein.

Using the returned sound signal and software configured for an analysis and production of electronic comparative sound graphs, such as FFT, electronic graphs are ascertained representative of the actual received sound broadcast by each headphone speaker.

The amplitude of the returned electronically transmitted sound samples “T” from the microphone, are plotted to electronic graphs as shown in FIG. 3, to compare the transmitted sound graph to a graph “R” of the correct reference sound signal, stored in memory, which the headphone speaker should produce for a calibrated test. The outgoing signal producing sound in the remote headphone speakers, is then adjusted if necessary, until a match of the returned sound graph “T” is achieved with the stored reference sound graph “R”. This calibration may be conducted with different tones and volume signals being communicated for play through headphone speakers in incremental decibel steps communicated to the user or patient's headphones, with each generating a returned graph of the sound produced and compared and adjusted until a calibration is achieved and all sounds for the test are sufficiently and accurately being reproduced on the patient or user's computer. Preferably, the incremental steps are performed in at least 5 db in order to ensure a suitable plot is obtained. Once the user or patient's system is confirmed to be producing accurate sound relative to the frequencies and volume levels communicated, the patient testing is initiated.

However, if the system employing software configured for the sound graph or other comparison to reference sounds detects a mistake or possible headphone mis-calibration either due to hardware error or user error during the calibration process, the user may be prompted to re-calibrate their headphones, calibrate a different sent of headphones, or to seek assistance from a testing professional.

As shown and described in FIG. 1, thereafter, using the testing software configured for administering standard psychometric test protocol, testing 16 begins. The user or patient at the remote location will be communicated tones or sounds in incremental steps to remotely test their hearing in a proper test with calibrated equipment. During the test, the tones are communicated in individual frequencies, through each respective speaker of the headphones to the adjacent ear of the user. During the testing the user or patient is asked by video or visual displayed queries the lowest volume tone of each communicated frequency they can hear from the generated frequency tones communicated at sequentially increasing volumes. Software configured to the task, ascertaining the keyed inputs from the user or patient, as to the lowest volume sound they discern at each communicated frequency tone, will ascertain any hearing impairment the communicating user or patient suffers.

As noted above, the results can be employed to inform the patient or user of the specifics of their hearing impairment. Or using the stored results and interfacing them with software configured to adjust the produced sound from the earphones, the user or patient can then by provided with “virtual” hearing aids by wearing the headphones. In such an example the headphones are adjusted to generate sound in a manner similar to a properly adjusted hearing aid so the patient or user can ascertain how corrected hearing will sound. The user would thus hear the improvement a hearing aid would provide before buying it.

Additionally as noted, for the family of the user or patient, a mate or friend of the person being tested, may wear the headphones, and simulated hearing problems may be communicated so they may listen and hear an actual simulation of how the patient or user actually hears. This allows them to better understand the problem and maybe even try to accommodate it in the future.

Finally, in another step if desired, an adjustment 18 of an existing hearing aid may be performed. Because the test administered is calibrated and meets industry norms, using software configured to communicate with the respective hearing aids from the remote computer, an adjustment to the software of the hearing aids, based on the test results, can be made. If the user or patient has hearing aids which are engaged for wired or wireless communication with the computer, such as with wireless Bluetooth® or wired USB engagement, using the stored test results, and appropriate adjustments based on such results, the hearing aids can be remotely adjusted to produce sound best adapted to accommodate the discerned hearing impairment of the wearer during the test.

In addition, as an option to the user, live or recorded video feed of the user performing the test can be communicated to the hearing professional during the test procedure. This will allow the professional to further aid the user in correctly performing the test, or to answer questions the user may have during test performance. This can be accomplished through the employment of video and audio recording means, such as a video or web-enable camera and microphone, which is in communication with users computer hardware or with the server over the network. As such the system will employ software adapted to the task of sending recorded or live video and audio over the network to the professional for immediate review, or for later review. Other means for direct lines of communication can include telephone or cellular network communication.

The method and components shown in the drawings and described in detail herein disclose arrangements of elements of particular construction, and configuration for illustrating preferred embodiments of structure of the present web based hearing testing device and method. It is to be understood, however, that elements of different construction and configuration, and using different steps and process procedures, and other arrangements thereof, other than those illustrated and described, may be employed for providing a surgical retrieval device and method in accordance with the spirit of this invention.

As such, while the present invention has been described herein with reference to particular embodiments thereof, a latitude of modifications, various changes and substitutions are intended in the foregoing disclosure, and will be appreciated that in some instance some features of the invention could be employed without a corresponding use of other features, without departing from the scope of the invention as set forth in the following claims. All such changes, alternations and modifications as would occur to those skilled in the art are considered to be within the scope of this invention as broadly defined in the appended claims.

Further, the purpose of the foregoing abstract of the invention, is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting, as to the scope of the invention in any way.

What is claimed is:

1. A system for administering a hearing test to be taken during a testing session by a user having a user computing device at a remote location which has access to a communication network, the system comprising:
a testing computing device in communication with said user computing device through said communication network;
a headphone operatively engaged with said user computing device to produce sound through a pair of headphone loudspeakers;
a microphone operatively engaged with said user computing device for electronically communicating an electronic signal of received sound from said microphone, to said user computing device;
said testing computing device having measuring software operable thereon, said measuring software configured to receive said electronic signal of received sound, from said user computing device, and calculate a background noise level at said remote location;
said testing computing device having calibrating software operable thereon, said calibrating software configured to communicate a first electronic signal over said network to said user computing device, said first electronic signal calculated to produce a test sound from each of said respective loudspeakers;
said test sound when received by said microphone generating a second electronic signal of received sound;
said calibrating software configured to receive said second electronic signal of received sound and generate an electronically storable test file representative of said test sound as received by said microphone;
said calibrating software configured to compare said test file for a match with a comparison file stored in computer memory in a first comparison, said comparison file being representative of an accurate broadcast of said test sound by a said loudspeaker;
said calibration software configured to adjust said first electronic signal communicated over said network to said user computing device, to generate a said test sound which results in a said match with said comparison file in the event a match is not ascertained in said first comparison;
testing software running on said testing computer device, said testing software configured to communicate a series of second electronic signals over said network to said user computing device;
said series of second electronic signals producing a series of different sounds which are communicated to respective ears of said user when wearing said headphones;
an input signal activated by said user on said user computing device, in response to inquiry as to a respective said ear of said user, hearing each of said series of different sounds;
said testing software configured to ascertain said user's hearing ability based on said input signal provided by said user in response to each said inquiry; and
whereby the sound produced by said loudspeakers and a sound level of said remote location can be determined in a calibration for said hearing test and said hearing test of said user's ears may be conducted with said user remotely located anywhere said user computing device can be engaged in an operative communication with said network.

2. The system for administering a hearing test of claim 1 additionally comprising:
said testing computing device having adjustment software running thereon, said adjustment software configured to conduct a review the results of said user's hearing test;
said adjustment software configured to communicate a third electronic signal, over said network, to said user computing device; and
said third electronic signal configured for receipt by a hearing aid placed in communication with said user computing device and an initiation of adjustments to sound produced by said hearing aid, based on said review of said user's hearing test.

3. A computer-assisted method of conducting a diagnostic hearing test upon a remotely located test subject having a user computing capable of electronic communication with a testing computer device over a network, comprising:
communicating instructions to said user how to input an input signal using said user computing device when requested;
communicating to said user to reply with a said input signal upon successfully engaging a microphone with said user computing device;
communicating to said user to reply with a said input signal upon successfully engaging a headphones having opposing loudspeakers, with said user computing device;
receiving a first electronic signal of received sound communicated to said user computing device, by said microphone;
employing software running on said testing computer to ascertain a background noise level from said signal of received sound;
if said background noise level is below a predetermined threshold, communicating a message to said user to reply with a said input signal upon successfully placing said microphone adjacent to a said loudspeaker which will subsequently produce a said test sound;
sequentially communicating a respective second electronic signal over said network to said user computing device for communication to respective said loudspeakers, said second electronic signal configured to produce a test sound from each of said respective loudspeakers in a sequence of respective test sounds;
receiving a second electronic signal of a received said test sound from said microphone over said network, for each said test sound broadcast by a said loudspeaker and received by said microphone;
employing software configured to receive each said second electronic signal of received said test sounds and thereafter generate an electronically storable test file, each said test file representative of a respective said second electronic signal of a received test sound;
employing calibrating software configured to compare each said test file with an electronically stored comparison file representing an accurate sound broadcast, by a said loudspeaker, of the respective said test sound represented by said test file;
if needed, adjusting said first electronic signal communicated over said network to said user computing device, to generate a said test sound from a respective said loudspeaker, which results in a said match with a respective said comparison file;
communicating instructions to said user how to input an input signal using said user computing device, in response to a hearing of testing sounds generated by a said loudspeaker which is communicated to a respective said ear of said user;
communicating a series of second electronic signals over said network to said user computing device, to produce a series said testing sounds to respective ears of said user;
monitoring and electronically storing said input signals from said user communicated in response to said testing sounds;
employing software running on said testing computer to ascertain said user's hearing ability based on said input signal provided by said user in response said testing sounds;
communicating instructions to said user how to input an input signal using said user computing device when requested; and
whereby said hearing test of said user's ears may be conducted with said user remotely located anywhere said user computing device may be engaged in an operative communication with said network, and the sound produced by said loudspeakers can be calibrated and a said threshold sound level of said remote location can be determined as acceptable for testing norms, prior to administering said hearing test to determine a said hearing ability, and.

4. The computer-assisted method of conducting a diagnostic hearing test of claim 3, additionally comprising the steps of:
employing software running upon said testing computing device to conduct a review the determined said hearing ability of said user;
based on said review, communicating an adjustment electronic signal, over said network, to said user computing device; and
allowing said user to communicate said adjustment electronic signal to a hearing aid placed in communication with said user computing device whereby said adjustment electronic signal adjusts the sound produced by said hearing aid, based on said review of said user's hearing test to enhance said user's hearing of said sound produced by said hearing aid.
